(19) United States
(12) Patent Application Publication
(10) Pub. No.: US 2019/0374590 A1
(43) Pub. Date: Dec. 12, 2019

(54) USE OF CORDYCEPS MILITARIS EXTRACT FOR TREATMENT OF RENAL DISEASE

(71) Applicant: Nan Pao Resins Chemical Co., Ltd., Tainan City (TW)

(72) Inventors: Chien-Tai Cheng, Tainan City (TW); Ting-Feng Wu, Tainan City (TW)

(21) Appl. No.: 16/423,595

(22) Filed: May 28, 2019

Related U.S. Application Data
(60) Provisional application No. 62/681,277, filed on Jun. 6, 2018.

(51) Int. Cl. A61K 36/068; A61K 31/045; A61K 31/565; A61K 31/7076

(52) U.S. Cl. CPC A61K 36/068; A61K 31/045; A61K 31/565; A61K 2236/53; A61K 2236/15; A61K 2236/17; A61K 31/7076

(57) ABSTRACT

A method for treating renal disease is provided in the present invention, and the method includes the step(s) of: administering a Cordyceps militaris extract to a subject in need thereof. Preferably, the Cordyceps militaris extract is manufactured by a method including the step(s) of: grinding a fruiting body of Cordyceps militaris into powders; mixing the Cordyceps militaris powders with ethanol to obtain a mixture; filtering the mixture to obtain a filtrate; and drying the filtrate to obtain the Cordyceps militaris extract.

### CROSS REFERENCE

This non-provisional application claims benefit of American Provisional Application No. 62/681,277, filed on Jun. 6, 2018, the content thereof is incorporated by reference herein.

### FIELD OF THE INVENTION

The present invention is related to pharmaceutical use of a Cordyceps militaris (CM) extract, and more particularly to use of a Cordyceps militaris extract for treating renal disease.

### BACKGROUND OF THE INVENTION

Podocytes are a group of cells existing in the Bowman's capsule of a kidney and wrapping around the glomerular capillaries. The Bowman's capsule can retain large molecules such as protein, red blood cells, or platelets and transport small molecules, such as water, salt, or sugar, to the Bowman's space so as to filter blood and thus form urine.

Podocytes have foot processes, and a slit diaphragm is formed between two adjacent foot processes. The slit diaphragm is responsible for blood filtering in the Bowman's capsule and has protein, e.g. nephrin, podocin, NEPH1, NEPH2, and CD2AP, for maintaining its biological function.

Therefore, downregulation of such protein level results in failure for filtering blood by the kidney.

Accordingly, it is desirable to develop a new medicine for the treatment of renal disease.

### SUMMARY OF THE INVENTION

The present invention is made based on the discovery that a Cordyceps militaris extract can promote nephrin protein level in human podocytes and suppress a-smooth muscle actin (α-SMA) protein level therein.

The present invention is further made based on the discovery that a Cordyceps militaris extract can reduce interstitial fibrosis, extracellular matrix accumulation, and glomerular fibrosis in a kidney of a mouse, especially in a kidney of a unilateral ureteral obstruction (UUO) mouse.

Therefore, an embodiment of the present invention provides a method for treating renal disease, and the method includes the step(s) of: administering a Cordyceps militaris extract to a subject in need thereof.

Preferably, the renal disease is chronic kidney disease (CKD), diabetic nephropathy, or renal fibrosis.

Preferably, the Cordyceps militaris extract is administered to the subject in 150-800 mg/kg of the subject.

Preferably, the Cordyceps militaris extract is an ethanol extract.

Preferably, the Cordyceps militaris extract is administered to the subject in a solution form obtained by mixing the Cordyceps militaris extract with a solvent.

Preferably, the Cordyceps militaris extract contains β-sitostenone. Preferably, the Cordyceps militaris extract contains cordycepin.

Preferably, the Cordyceps militaris extract at least contains β-sitostenone and cordycepin.

Preferably, the Cordyceps militaris extract is manufactured by a method including the step(s) of: grinding a fruiting body of Cordyceps militaris into powders; mixing the Cordyceps militaris powders with ethanol to obtain a mixture; filtering the mixture to obtain a filtrate; and drying the filtrate to obtain the Cordyceps militaris extract.

Preferably, the Cordyceps militaris powders and the ethanol are mixed in a weight to volume ratio of 1:5 to 1:15.

Another embodiment of the present invention provides a method for promoting nephrin protein level in a cell, and the method includes the step(s) of: incubating a cell in need thereof with a Cordyceps militaris extract.

Preferably, the cell is a podocyte.

Preferably, the Cordyceps militaris extract is an ethanol extract.

Preferably, the Cordyceps militaris extract is incubated with the cell in a solution form obtained by mixing the Cordyceps militaris extract with a solvent.

Preferably, the Cordyceps militaris extract contains β-sitostenone.

Preferably, the Cordyceps militaris extract contains cordycepin.

Preferably, the Cordyceps militaris extract at least contains β-sitostenone and cordycepin.

Preferably, the Cordyceps militaris extract is manufactured by a method including the step(s) of: grinding a fruiting body of Cordyceps militaris into powders; mixing the Cordyceps militaris powders with ethanol to obtain a mixture; filtering the mixture to obtain a filtrate; and drying the filtrate to obtain the Cordyceps militaris extract.

Preferably, the Cordyceps militaris powders and the ethanol are mixed in a weight to volume ratio of 1:5 to 1:15.

Another yet embodiment of the present invention provides a method for inhibiting α-smooth muscle actin protein level in a cell, and the method includes the step(s) of: incubating a cell in need thereof with a Cordyceps militaris extract.

Preferably, the cell is a podocyte.

Preferably, the Cordyceps militaris extract is an ethanol extract.

Preferably, the Cordyceps militaris extract is incubated with the cell in a solution form obtained by mixing the Cordyceps militaris extract with a solvent.

Preferably, the Cordyceps militaris extract contains β-sitostenone.

Preferably, the Cordyceps militaris extract contains cordycepin.

Preferably, the Cordyceps militaris extract at least contains β-sitostenone and cordycepin.

Preferably, the Cordyceps militaris extract is manufactured by a method including the step(s) of: grinding a fruiting body of Cordyceps militaris into powders; mixing the Cordyceps militaris powders with ethanol to obtain a mixture; filtering the mixture to obtain a filtrate; and drying the filtrate to obtain the Cordyceps militaris extract.

Preferably, the Cordyceps militaris powders and the ethanol are mixed in a weight to volume ratio of 1:5 to 1:15.

### BRIEF DESCRIPTION OF THE DRAWINGS

FIG. 1 is a bar graph illustrating nephrin protein level in AB8/13 human podocytes after the treatment with various substances;

FIG. 2 is a bar graph illustrating a-smooth muscle actin protein level in AB8/13 human podocytes after the treatment with various substances;

FIG. 3 is a bar graph illustrating nephrin protein level in AB8/13 human podocytes after the treatment with various substances;

FIG. 4 is a bar graph illustrating renal interstitial fibrosis level in mice;

FIG. 5 is a bar graph illustrating renal extracellular matrix accumulation level in mice; and

FIG. 6 is a bar graph illustrating glomerular fibrosis level in mice.

### DETAILED DESCRIPTION OF THE INVENTION

The detailed description and preferred embodiments of the invention will be set forth in the following content, and provided for people skilled in the art so as to understand the characteristics of the invention.

EXTRACTION FROM CORDYCEPS MILITARIS

After a fruiting body of Cordyceps militaris was ground into powders, 50 g of the Cordyceps militaris powders and 95% aqueous ethanol solution were mixed in a weight to volume ratio of 1:10. After the obtained mixture was shaken at a room temperature for 18 hours, the supernatant was taken to be filtered. Finally, the filtered liquid was dried in a vacuum to obtain a Cordyceps militaris extract (13.16 g) as a brown syrup.

In order to perform the bio-assay as described below and/or other bio-assays not provided therein, the obtained extract was dissolved in a solvent, e.g. dimethyl sulfoxide (DMSO), phosphate buffered saline (PBS), or distilled deionized water (dd water). It is noted that the concentration described below is a ratio of the weight of the Cordyceps militaris extract to the total volume of the Cordyceps militaris extract and the solvent.

Additionally, according to the high-performance liquid chromatography (HPLC) result, the extract at least contains β-sitostenone and cordycepin.

IN VITRO ASSAY

Firstly, AB8/13 human podocytes were incubated in a 33□ incubator containing 5% carbon dioxide until they grew to a confluence of 80%. The cells were transferred to a 37.5□ incubator containing 5% carbon dioxide to be incubated for 14 days so that the cells were differentiated. After which, the differentiated cells were incubated with culture medium containing various substances for 6 days. Finally, total protein of the differentiated cells treated with various substances was extracted for Western blotting assay.

As stated in Diabetes, 2011 Jun; 60(6): 1779-1788, transforming growth factor-β1 (TGF-β1) is greatly expressed in the renal tissue of a diabetic nephropathy patient. This paper further has reported that transforming growth factor-β1 can dedifferentiate differentiated podocytes and induce the disappearance of pseudopods. As stated in Cell Tissue Res. 2012 Jan; 347(1): 141-54, transforming growth factor-β1 participates in renal fibrosis, renal parenchyma degeneration and loss of function associated with chronic kidney disease. For the above reasons, podocytes treated with transforming growth factor-β1 can be used as a cell culture model for renal disease.

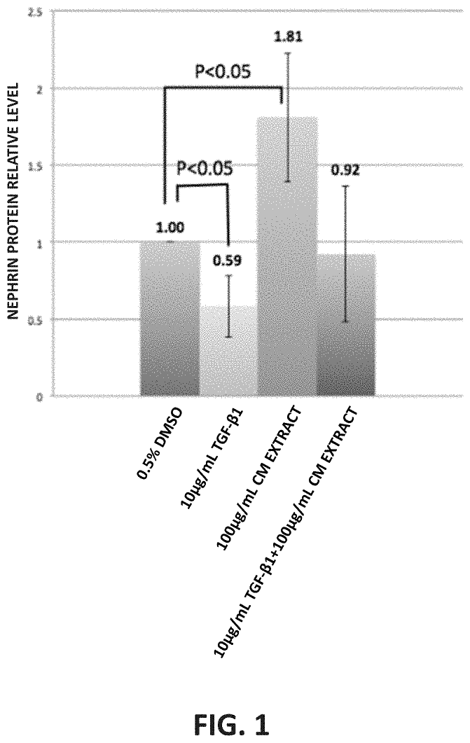

As shown in FIG. 1, as compared to dimethyl sulfoxide, transforming growth factor-β1 can downregulate the nephrin protein level in AB8/13 human podocytes, and the Cordyceps militaris extract can upregulate such protein level in AB8/13 human podocytes. Furthermore, the Cordyceps militaris extract and transforming growth factor-β1 can together maintain the nephrin protein level in AB8/13 human podocytes, which indicates that the Cordyceps militaris extract can compensate for the low nephrin protein level resulted from transforming growth factor-β1. As described in “BACKGROUND OF THE INVENTION”, nephrin is the protein for maintaining a slit diaphragm; that is, the Cordyceps militaris extract exhibits the function of maintaining renal slit diaphragms.

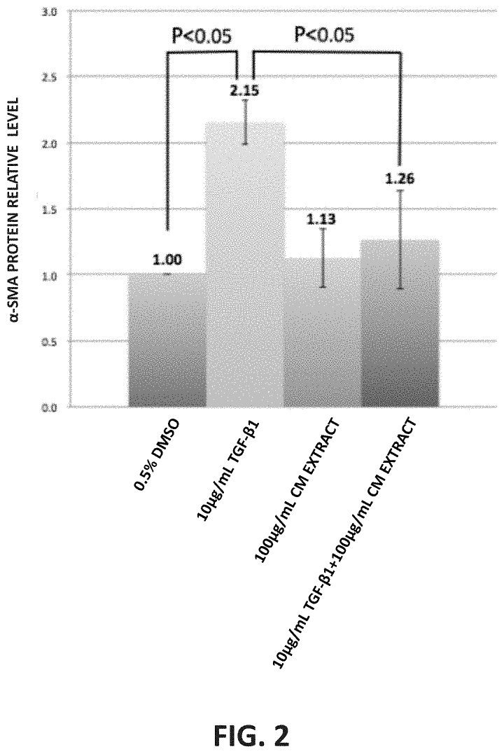

As shown in FIG. 2, as compared to dimethyl sulfoxide, transforming growth factor-β1 can upregulate the a-smooth muscle actin protein level in AB8/13 human podocytes; the Cordyceps militaris extract can maintain such protein level in AB8/13 human podocytes under the condition that the cells are treated with or without transforming growth factor-β1. This indicates that the Cordyceps militaris extract can lower the high a-smooth muscle actin protein level resulted from transforming growth factor-β1. Generally speaking, α-smooth muscle actin can transform differentiated podocytes into fibroblasts derived from undifferentiated podocytes. Therefore, the Cordyceps militaris extract can maintain the differentiated podocytes so as to exhibit the function of maintaining renal slit diaphragms.

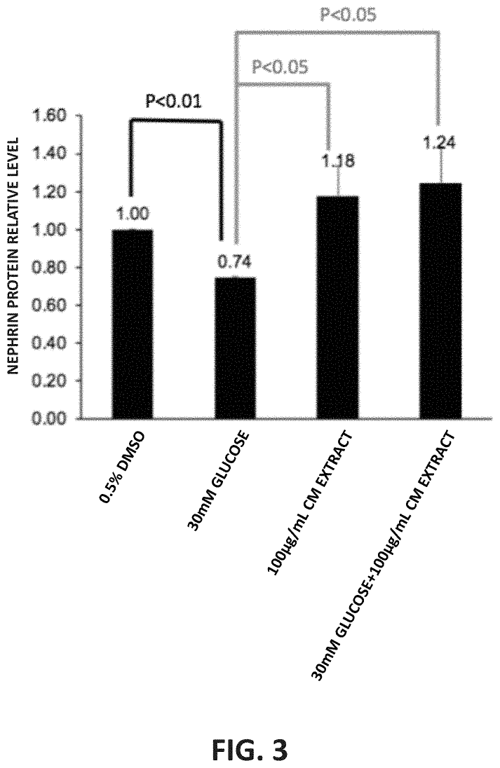

As shown in FIG. 3, as compared to dimethyl sulfoxide, high glucose can downregulate the nephrin protein level in AB8/13 human podocytes; the Cordyceps militaris extract not only can upregulate the protein level in AB8/13 human podocytes, but also can compensate for the low nephrin protein level resulted from high glucose.

As described above, the Cordyceps militaris extract can promote nephrin protein level and suppress a-smooth muscle actin protein level in vitro; therefore, the Cordyceps militaris extract has the potential for treating the renal disease, especially diabetic nephropathy and chronic kidney disease.

IN VIVO ASSAY

Mice were grouped into a sham control group, a unilateral ureteral obstruction only group, a unilateral ureteral obstruction combined with phosphate buffered saline treatment group, and a unilateral ureteral obstruction combined with Cordyceps militaris extract treatment group. Then, unilateral ureteral obstruction surgery was performed on the mice of the latter three groups. After two days, the mice of the unilateral ureteral obstruction combined with phosphate buffered saline treatment group and those of the unilateral ureteral obstruction combined with Cordyceps militaris extract treatment group were orally treated with the corresponding substances. On the 7th day after the substance treatment, the kidneys of all mice were sliced and stained for rating the renal interstitial fibrosis level, the renal extracellular matrix accumulation level, and the glomerular fibrosis level. The score for rating ranged from 1 to 5; if the score was 5, it indicated the renal interstitial fibrosis, the renal extracellular matrix accumulation, or the glomerular fibrosis was present in the severest manner, and if the score was 1, it indicated the renal interstitial fibrosis, the renal extracellular matrix accumulation, or the glomerular fibrosis was present in the mildest manner.

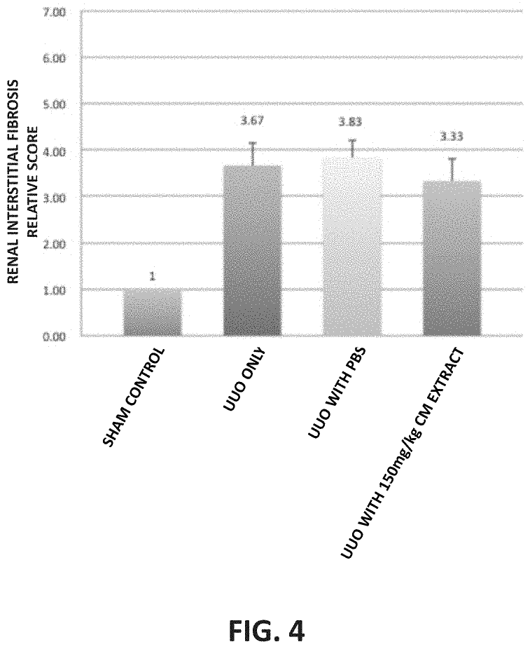

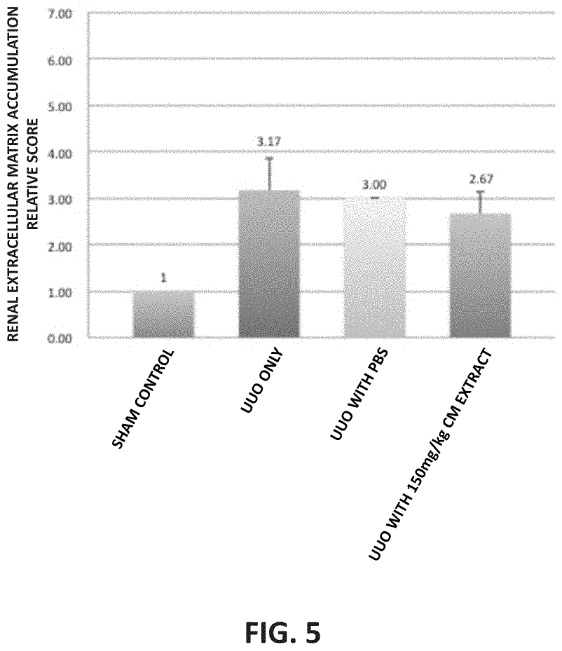

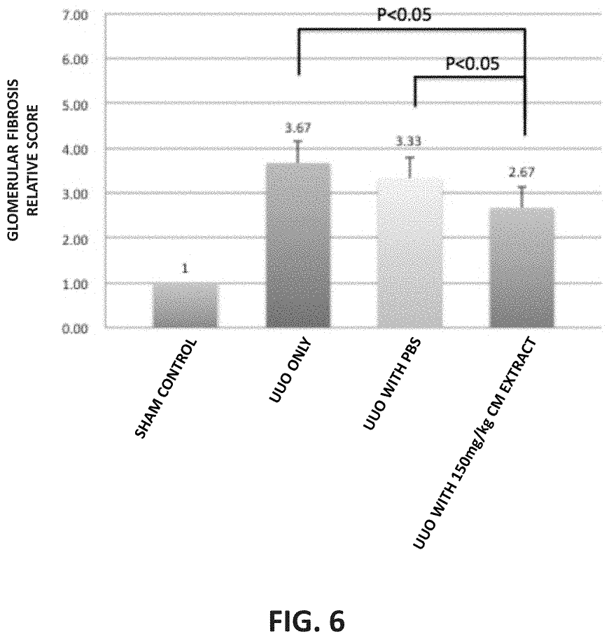

As shown in FIGS. 4-6, unilateral ureteral obstruction surgery can lead to the renal interstitial fibrosis, the renal extracellular matrix accumulation, and the glomerular fibrosis of mice. As compared to phosphate buffered saline, the Cordyceps militaris extract can retard the high renal interstitial fibrosis level, the high renal extracellular matrix accumulation level, and the high glomerular fibrosis level result from unilateral ureteral obstruction.

As described above, the Cordyceps militaris extract can cure renal interstitial fibrosis, renal extracellular matrix accumulation, and glomerular fibrosis in vivo; therefore, the Cordyceps militaris extract has the potential for treating the renal disease, especially renal fibrosis.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for treating renal disease comprising:
administering a Cordyceps militaris extract to a subject in need thereof.

2. The method as claimed in claim 1, wherein the renal disease is chronic kidney disease (CKD), diabetic nephropathy, or renal fibrosis.

3. The method as claimed in claim 1, wherein the Cordyceps militaris extract is administered to the subject in 150-800 mg/kg of the subject.

4. The method as claimed in claim 1, wherein the Cordyceps militaris extract is an ethanol extract.

5. The method as claimed in claim 4, wherein the Cordyceps militaris extract is manufactured by a method comprising:
grinding a fruiting body of Cordyceps militaris into powders;
mixing the Cordyceps militaris powders with ethanol to obtain a mixture;
filtering the mixture to obtain a filtrate; and
drying the filtrate to obtain the Cordyceps militaris extract.

6. The method as claimed in claim 5, wherein the Cordyceps militaris powders and the ethanol are mixed in a weight to volume ratio of 1:5 to 1:15.

7. The method as claimed in claim 1, wherein the Cordyceps militaris extract at least includes β-sitostenone.

8. The method as claimed in claim 1, wherein the Cordyceps militaris extract at least includes cordycepin.

9. A method for promoting nephrin protein level in a cell comprising:
incubating a cell in need thereof with a Cordyceps militaris extract.

10. The method as claimed in claim 9, wherein the Cordyceps militaris extract is an ethanol extract.

11. The method as claimed in claim 10, wherein the Cordyceps militaris extract is manufactured by a method comprising:
grinding a fruiting body of Cordyceps militaris into powders;
mixing the Cordyceps militaris powders with ethanol to obtain a mixture;
filtering the mixture to obtain a filtrate; and
drying the filtrate to obtain the Cordyceps militaris extract.

12. The method as claimed in claim 11, wherein the Cordyceps militaris powders and the ethanol are mixed in a weight to volume ratio of 1:5 to 1:15.

13. The method as claimed in claim 9, wherein the Cordyceps militaris extract at least includes β-sitostenone or cordycepin.

14. The method as claimed in claim 9, wherein the cell is a podocyte.

15. A method for inhibiting α-smooth muscle actin protein level in a cell comprising:
incubating a cell in need thereof with a Cordyceps militaris extract.

16. The method as claimed in claim 15, wherein the Cordyceps militaris extract is an ethanol extract.

17. The method as claimed in claim 16, wherein the Cordyceps militaris extract is manufactured by a method comprising:
grinding a fruiting body of Cordyceps militaris into powders;
mixing the Cordyceps militaris powders with ethanol to obtain a mixture;
filtering the mixture to obtain a filtrate; and
drying the filtrate to obtain the Cordyceps militaris extract.

18. The method as claimed in claim 17, wherein the Cordyceps militaris powders and the ethanol are mixed in a weight to volume ratio of 1:5 to 1:15.

19. The method as claimed in claim 15, wherein the Cordyceps militaris extract at least includes β-sitostenone or cordycepin.

20. The method as claimed in claim 15, wherein the cell is a podocyte.
